(19) United States
(12) Patent Application Publication
(10) Pub. No.: US 2022/0218979 A1
(43) Pub. Date: Jul. 14, 2022

(54) NEUROSTIMULATORS TO IMPROVE MALE ORGASM AND SYSTEMS AND METHODS FOR USE

(71) Applicant: THE BOARD OF TRUSTEES OF THE LELAND STANFORD JUNIOR UNIVERSITY, Stanford, CA (US)

(72) Inventor: Michael L. Eisenberg, Stanford, CA (US)

(21) Appl. No.: 17/707,854

(22) Filed: Mar. 29, 2022

Related U.S. Application Data
(63) Continuation of application No. PCT/US2020/053612, filed on Sep. 30, 2020.
(60) Provisional application No. 62/908,502, filed on Sep. 30, 2019.

(51) Int. Cl. A61N 1/04; A61N 1/36; A61F 5/41

(52) U.S. Cl. CPC A61N 1/0456; A61N 1/0484; A61F 2005/418; A61N 1/36031; A61F 5/41; A61N 1/36007

(57) ABSTRACT

Devices are provided for inducing orgasm that include a ring or other wearable component sized for placement around a penis, the ring including one or more electrodes for delivering electrical stimulae to the penis, and a neurostimulator module. The neurostimulator module may include a controller coupled to the one or more electrodes for controlling delivery of the electrical stimulae, and a communications interface coupled to the controller for receiving external commands related to delivery of the electrical stimulae.

### RELATED APPLICATION DATA

The present application is a continuation of co-pending International Application Serial No. PCT/US2020/053612, filed Sep. 30, 2020, which claims benefit of U.S. provisional application Ser. No. 62/908,502, filed Sep. 30, 2019, the entire disclosures of which are expressly incorporated by reference herein.

### FIELD OF THE INVENTION

The present invention relates to devices, systems, and methods for inducing orgasm in men and, more particularly, to a wearable penile nerve stimulator device for expediting or inducing orgasm and to systems and methods for using such devices.

### BACKGROUND

Delayed orgasm (“DO”), also called delayed ejaculation, anorgasmia, and retarded ejaculation, affects five to ten percent (5-10%) of men leading to anxiety and distress. Individuals suffering from DO generally experience persistent or recurrent difficulty, delay in, or absence of attaining orgasm after sufficient sexual stimulation causing personal distress. As a result, afflicted individuals may suffer from decreased self-esteem; feelings of inadequacy, failure, and negativity; anxiety about sex; decreased sexual pleasure; male infertility; low libido; conflicts in relationships, and/or other side effects, potentially leading to the individuals avoiding intimacy with others and/or experiencing difficulties in their personal relationships.

Currently no effective solutions exist for many subjects experiencing DO. Subjects may consult their urologist and/or a sex therapist, e.g., to receive one or more of cognitive therapy, physical therapy, medication, and/or other treatments. However, for many subjects, these treatments are ineffective. Implantable nerve stimulators have been suggested, but such stimulators require surgery to implant and remove them.

Therefore, devices and methods that facilitate inducing male orgasm would be useful.

### SUMMARY

The present invention is directed to devices, systems, and methods for inducing orgasm in men and, more particularly, to wearable penile nerve stimulator devices for expediting or inducing orgasm and to systems and methods for using such devices, e.g., to improve sexual satisfaction in men experiencing DO. In addition, the devices, systems, and methods herein may be used to treat other conditions, such as premature ejaculation, rehabilitation after pelvic cancer/injury, and the like, or to assist in diagnostic procedures, such as electromyography (“EMG”) and the like, involving the penis.

Generally, a wearable penile nerve stimulator is provided, which may target the dorsal genital nerve and/or other nerves or regions of the penis, to expedite and/or improve male orgasm. In an exemplary embodiment, the device may include a reusable component or module including a neurostimulator, a rechargable battery, and a communication unit (e.g., a remote control, mobile phone, tablet, or other electronic device). The reusable module may connect to a disposable band or other wearable component allowing direct skin contact, e.g., targeting of the dorsal penile nerve. The band may be provided in different sizes and may be sufficiently durable to allow for multiple uses before disposal. Alternatively, the neurostimulator module may be permanently integrated into the wearable component or may be separate from the wearable component, e.g., within a remote control that may be connected to the wearable component by one or more cables and/or wirelessly, and the device may be used indefinitely, e.g., with the wearable component cleaned after each use. The wearable component may work on its own or be used with conducting gel/liquid.

In accordance with an exemplary embodiment, a device is provided for inducing orgasm that includes a wearable component sized for placement on or around a penis, the wearable component including one or more electrodes for delivering electrical stimulae to the penis, and a neurostimulator module. The neurostimulator module may include a controller coupled to the one or more electrodes for controlling delivery of the electrical stimulae, and a communications interface coupled to the controller for receiving external commands related to delivery of the electrical stimulae. In exemplary embodiments, the wearable component may include one or more rings, straps, bands, a tubular sleeve or body, a clamp, and the like. Optionally, the wearable component and/or module may include one or more sensors for monitoring one or more physical parameters of the user.

In accordance with another embodiment, a system is provided for inducing or expediting orgasm that includes a wearable component sized for placement on and/or around a penis, the wearable component including one or more electrodes for delivering electrical stimulae to the penis, and a neurostimulator module. The neurostimulator module may include a controller coupled to the one or more electrodes for controlling delivery of the electrical stimulae, and a communications interface coupled to the controller for receiving external commands related to delivery of the electrical stimulae. The system may also include an electronic device, e.g., a remote control, mobile phone, tablet computer, and the like, including a user interface and a communications interface to allow a user to send commands to the neurostimulator module, e.g., to activate the controller and/or modify the electrical stimulae being delivered.

In accordance with still another embodiment, a method is provided for inducing or expediting orgasm that includes placing a wearable device on a penis to position one or more electrodes at a desired region on the penis, and activating a neurostimulator on the wearable device to deliver electrical stimulae to the desired region via the one or more electrodes.

In accordance with yet another embodiment, a method is provided for treating a patient after pelvic cancer treatment/pelvic or genital injury that includes placing a wearable device on or adjacent a penis to position one or more electrodes at a desired region relative to the penis; and activating a neurostimulator on the wearable device to deliver electrical stimulae to the desired region via the one or more electrodes to stimulate or rehabilitate penile or pelvic sensory pathways.

In accordance with still another embodiment, a method is provided for treating premature ejaculation that includes placing a wearable device on or adjacent a penis to position one or more electrodes at a desired region relative to the penis; and activating a neurostimulator on the wearable device to deliver electrical stimulae to the desired region via the one or more electrodes.

In accordance with another embodiment, a device is provided for inducing or expediting orgasm that includes a wearable component sized for placement around a penis, the wearable component including one or more electrodes for delivering electrical stimulae to the penis; and a neurostimulator module comprising a housing removably connectable to the wearable component, a controller coupled to the one or more electrodes when the housing is connected to the wearable component for controlling delivery of the electrical stimulae, and a communications interface coupled to the controller for receiving external commands related to delivery of the electrical stimulae.

In accordance with still another embodiment, a device is provided for inducing or expediting orgasm that includes a wearable component sized for placement around a penis, the wearable component including one or more electrodes for delivering electrical stimulae to the penis; and a neurostimulator module comprising a housing permanently mounted to the wearable component, a controller coupled to the one or more electrodes for controlling delivery of the electrical stimulae, and a communications interface coupled to the controller for receiving external commands related to delivery of the electrical stimulae.

In accordance with yet another embodiment, a device is provided for inducing or expediting orgasm that includes a wearable component sized for placement around a penis, the wearable component including one or more electrodes for delivering electrical stimulae to the penis; a remote control device comprising a housing separate from the wearable component; and a cable including one or more wires extending between the housing and the wearable component, wherein the remote control comprises a neurostimulator module including a controller coupled to the one or more electrodes via the one or more wires for controlling delivery of the electrical stimulae.

In accordance with another embodiment, a system is provided for inducing or expediting orgasm that includes a wearable component sized for placement around a penis, the wearable component including one or more electrodes for delivering electrical stimulae to the penis; and a neurostimulator module including a controller coupled to the one or more electrodes for controlling delivery of the electrical stimulae, and a communications interface coupled to the controller for receiving external commands related to delivery of the electrical stimulae. The system also includes an electronic device comprising a user interface and communications interface to allow a user to send commands to the neurostimulator module, e.g., wirelessly or via a cable connected to the neurostimulator module.

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It is emphasized that, according to common practice, the various features and design elements of the drawings are not to-scale. On the contrary, the dimensions of the various features and design elements are arbitrarily expanded or reduced for clarity. Included in the drawings are the following figures:

FIGS. 1A and 1B are perspective views of an exemplary embodiment of a wearable neurostimulator device including a ring including a pair of electrodes and a neurostimulator module connected the ring and electrodes.

FIG. 1C is a schematic showing exemplary components that may be included in the neurostimulator module of the wearable device of FIGS. 1A and 1B.

FIGS. 2A and 2B are cross-sectional views of male genitals showing the wearable neurostimulator device of FIGS. 1A and 1B worn on the penis.

FIGS. 3A-3F show examples of alternative embodiments of wearable components that may be included in a neurostimulator device.

FIGS. 4A-4C show examples of neurostimulator devices that include a remote control separate from a wearable component.

### DETAILED DESCRIPTION OF THE EXEMPLARY EMBODIMENTS

Before the exemplary embodiments are described, it is to be understood that the invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

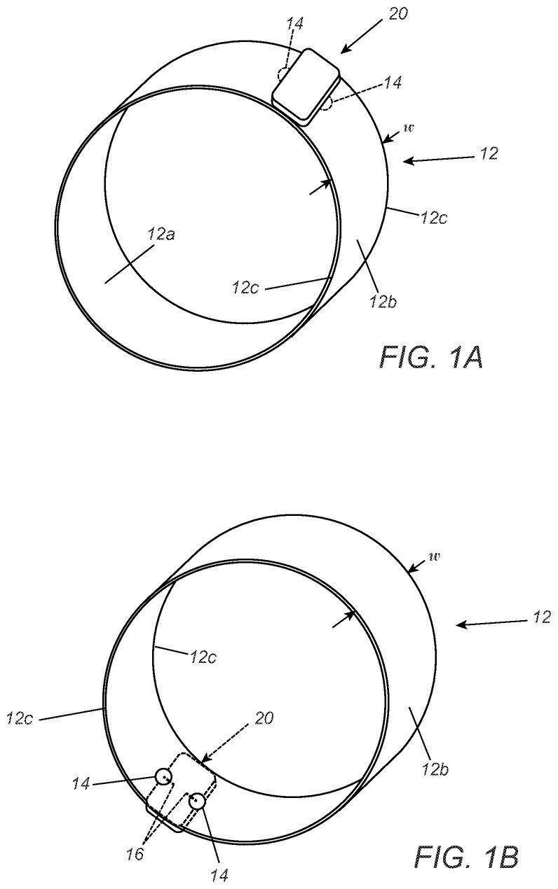

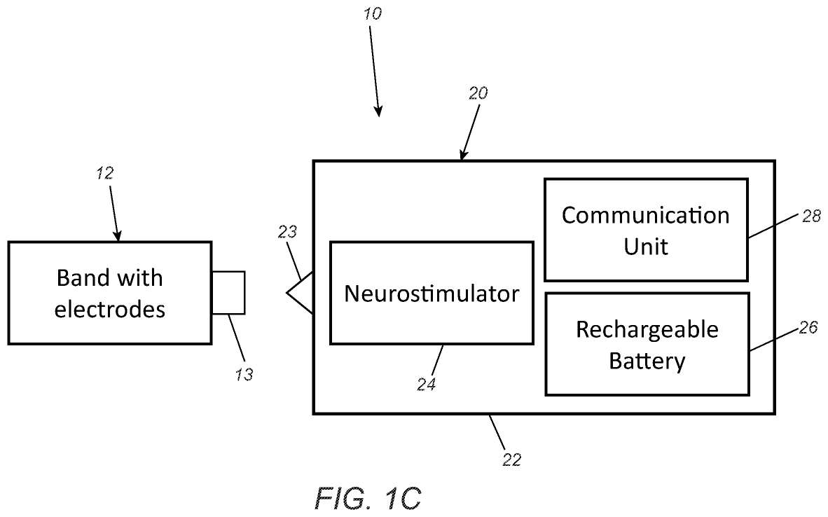

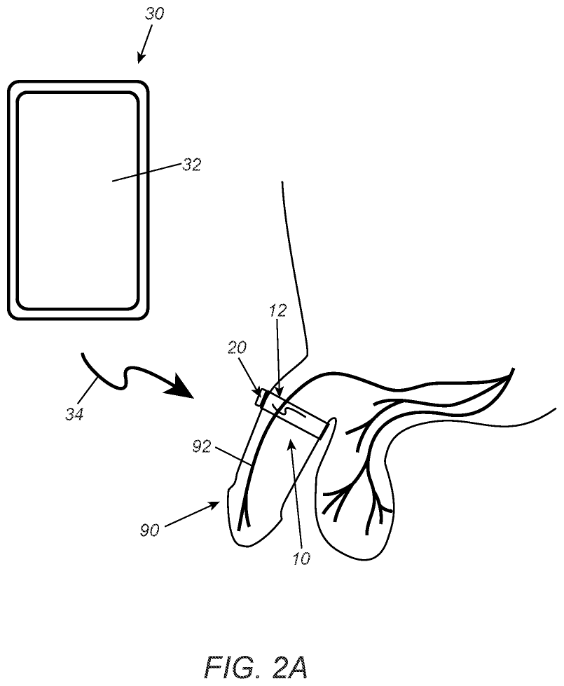

Turning to the drawings, FIGS. 1A-1C show an exemplary embodiment of a device 10 for inducing orgasm in men that generally includes a wearable ring or band 12 sized for placement around a penis 90 (not shown, see FIG. 2A) and a neurostimulator or control module 20. The ring 12 may carry one or more electrodes or other conductive pads, e.g., a pair of pads 14 as shown located on an inner surface 12a of the ring 12. Optionally, the ring 12 may be provided in a range of sizes such that an individual user may use a ring of an appropriate size for their penis.

In exemplary embodiments, the ring 12 may be formed from flexible biocompatible materials to increase comfort during use, e.g., to facilitate placement and removal. For example, the ring 12 may be formed from an enclosed band of silicone or other elastomeric or polymeric materials that may be sufficiently elastic to stretch slightly to accommodate placement and removal and/or to adjust in size when the penis 90 is stimulated. Alternatively, the ring 12 may be formed from inelastic material, e.g., PVC and/or other plastic or non-electrically conductive materials, having a fixed diameter or other shape, e.g., between about 20-50 mm, between about 20-30 mm, between about 30-40 mm, or between about 40-50 mm. As shown in FIGS. 1A and 1B, the ring 12 is an enclosed loop having a width “W,” e.g., between about 5-30 mm, defining an inner surface 12a, an outer surface 12b, and opposing annular side edges 12c.

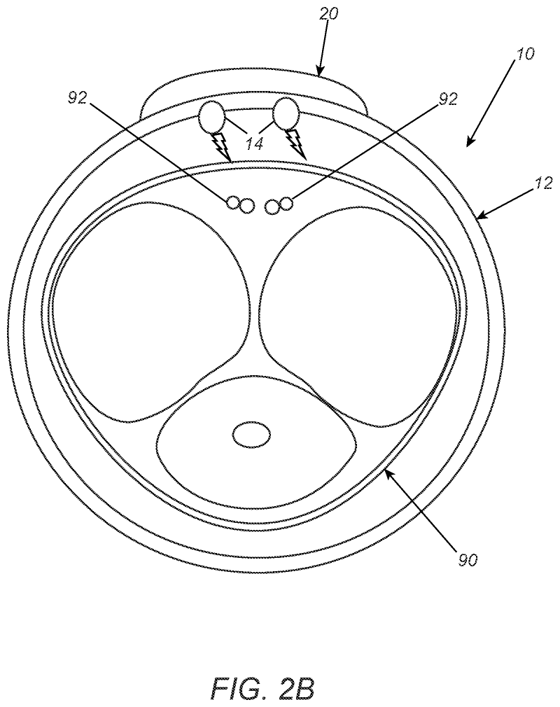

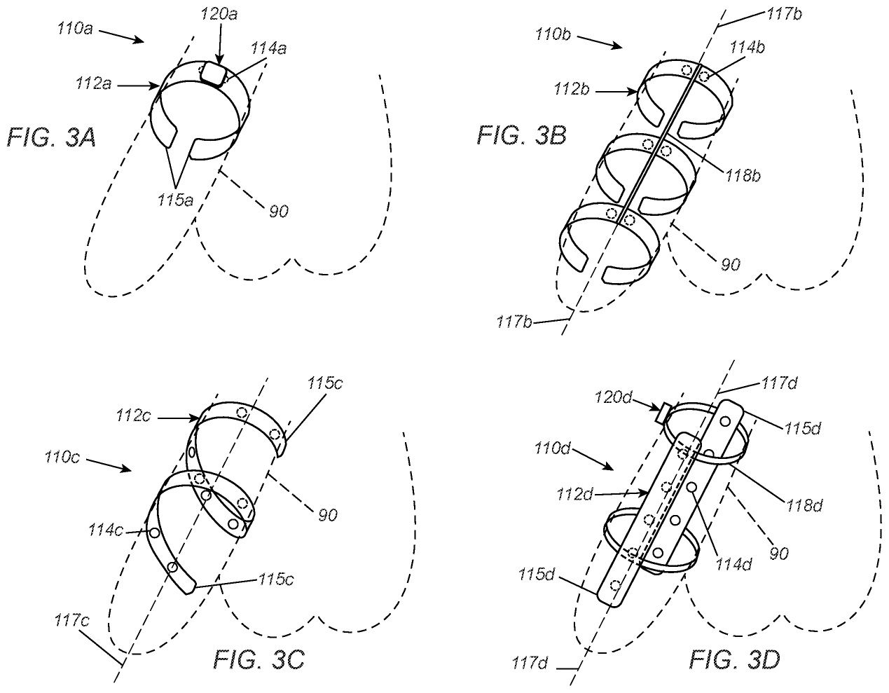

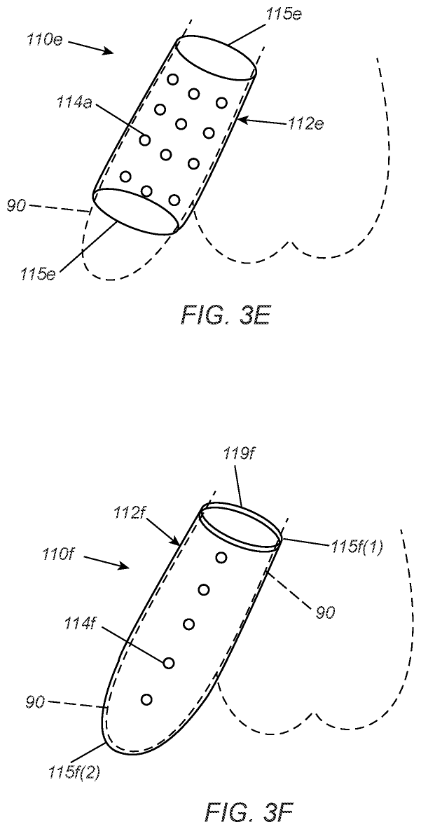

Alternatively, a ring may be provided that is not completely enclosed. For example, FIG. 3A shows an example of a neurostimulator device 110a that includes a ring 112a and control module 120a coupled to electrodes 114a, generally similar to the previous embodiments. In this case, however, the ring 112a has a “C” shaped cross-section that includes ends 115a that may be spaced apart one another and/or that may partially overlap in a relaxed state. Optionally, in this alternative, the ends 115a may be rounded or otherwise shaped to minimize discomfort when the ring is worn. In addition, such a “C” shaped ring 112a may be elastic, e.g., biased to a relaxed diameter or state, yet radially expandable, may be inelastic, e.g., set at a fixed diameter, or may be malleable, e.g., such that the ends 115a may be forced apart to increase the diameter and/or overlapped or otherwise directed to towards one another to reduce the diameter, e.g., to wrap the ring 112a at least partially around a penis 90 (shown in phantom).

In another alternative, shown in FIG. 3B, a device 110b may be provided that includes a plurality of rings 112b, each having a “C” shaped (or circular, not shown) cross-section, that are spaced apart from one another along a central longitudinal axis 117b. The rings 112b may be permanently connected together by one or more axial supports 118b (one shown) to maintain their spacing. Each of the rings 112b may include one or more electrodes, e.g., two electrodes 114b adjacent one another for delivering stimulae to the underlying dorsal nerves (not shown) or a plurality of electrodes around the inner surface, similar to other embodiments herein. The support(s) 118b may include one or more wires or other leads (not shown), e.g., embedded within the support(s) 118b to couple the electrodes 114b to the neurostimulator module 120b, e.g., for delivering stimulae simultaneously, sequentially, and/or in other patterns, as described elsewhere herein. The support(s) 118b may be flexible, rigid, or malleable, similar to other embodiments herein. In addition, the rings 112b may be elastic, as described above, or may be malleable, e.g., such the rings 112b may be opened, positioned around the penis 90, and then deformed to clamp or otherwise secure the rings 112b around the penis 90.

In yet another alternative, shown in FIG. 3C, a neurostimulator device 110c may be provided that includes a helical ring 112c that spirals one or more times around a central axis 117c between opposite ends 115c of the ring 112b, which may also be elastic, inelastic, or malleable, similar to the previous embodiments. In this alternative, a plurality of electrodes 114b may be provided, as desired, spaced apart from one another around the circumference and/or along the length of the helical ring 112b, e.g., a pair of electrodes adjacent one another on the same side of each loop for delivering stimulae to the underlying dorsal nerves (not shown), or spaced apart evenly or otherwise around the inner surface between the ends 115c.

In still another alternative, shown in FIG. 3D, a neurostimulator device 110d may be provided that includes one or more longitudinal bands 112d that are connected together by one or more annular or “C” shaped supports 118d, e.g., to space the bands 112d around central axis 117d. For example, with two bands 112d, as shown, the bands 112d may be attached to the supports 118d opposite one another for placement on opposite sides of a penis 90. Each band 112d may include one or more electrodes, e.g., a plurality of electrodes 114d spaced apart from one another at least partially along the length of the band 112d. One or more leads (not shown) in the bands 112d and/or supports 118d couple the electrodes 114d to the neurostimulator module 120d for delivering the electrical stimulae.

In additional alternatives, shown in FIGS. 3E and 3F, a neurostimulator device 110e, 110f may be provided that includes one or more electrodes 114e, 114f on a tubular sleeve 112e, 112f, e.g., formed from elastic or inelastic material such as those described above. For example, as shown in FIG. 3E, the sleeve 112e may include open ends 115e or, as shown in FIG. 3F, the sleeve 112f may include an open end 115f(1) and a closed end 115f(2), e.g., configured similar to a condom (not shown), that may be positioned over the penis 90 such that the electrode(s) 114e, 114f are located at desired locations. Optionally, as shown in FIG. 3F, the sleeve 112f may include an annular collar 119f on the open end 115f(1), e.g., to facilitate sliding the sleeve 112f into place. Alternatively, a sleeve may be formed as a sheet (not shown) that may be rolled around the penis and longitudinal edges of the sheet may be removably attached together to secure the sleeve, e.g., using low tack adhesive, hook and eye fasteners, elastic, self-cinching/friction, and the like. In still another alternative, a mesh or net structure may be provided, e.g., having a tubular shape (not shown), that may be positioned over the penis. In a further alternative, one or more electrodes may be provided on an adhesive strip, band, or patch (also not shown), which may be removably adhered to a desired location on or adjacent the user's penis. In these alternatives, electrodes may be spaced apart from one another as desired, e.g., around a circumference and/or along a length of the wearable component.

Returning to FIGS. 1A and 1B, the electrodes or pads 14 may be formed from biocompatible, conductive material, e.g., formed to be flush with the inner surface 12a of the ring 12 (or other wearable component, as described elsewhere herein) and/or otherwise shaped and/or mounted to the ring 12 to maximize comfort for the user using the device 10 while ensuring the electrodes 14 contact the user's skin. For example, the electrodes 14 may be molded into the material of the ring 12, may be permanently attached to the inner surface 12a, e.g., by one or more of bonding with adhesive, fusing, heat sealing, inserting into recesses, and the like. The ring 12 may include one or more leads 16 communicating with the electrodes 14, e.g., embedded within the ring 12 to deliver electrical signals to the electrodes 14 from the neurostimulator module 20.

Optionally, the ring 12 (or other wearable components herein) may include one or more visual markers (not shown), e.g., positioned at one or more locations on the outer surface 12b, side edges 12c, and/or otherwise around the circumference of the ring 12, which may facilitate the user orienting the ring 12 to position the electrodes 14 adjacent a desired target region. For example, as shown in FIG. 2B, a pair of electrodes 14 are provided immediately adjacent one another for stimulating the dorsal nerves 92 of the penis 90 and the ring 12 may include a marker (not shown) on the outer surface opposite the electrodes such that the user may orient the marker anteriorly to position the electrodes 14 adjacent the dorsal nerves. Alternatively, a plurality of electrodes may be provided on the ring 12 that are distributed around the circumference of the inner surface 12a, e.g., in one or more rows or other patterns spaced substantially uniformly apart from one another (not shown), to deliver the electrical stimulae uniformly to the underlying region.

The neurostimulator module 20 may be removably mountable to the ring 12 (or the other wearable components described herein), e.g., such that the neurostimulator module 20 may be reusable when the ring 12 wears or deteriorates and must be replaced. For example, as shown in FIG. 1C, the neurostimulator 20 module may include an external housing 22 containing and/or protecting the internal components, e.g., a controller or neurostimulator 24, a battery or other power source 26, and a communications interface 28. The housing 22 may provide a fluid-tight package, e.g., such that the neurostimulator module 20 and/or entire device 10 may be cleaned after use, as desired, without damaging circuits of the module 20.

Optionally, the ring 12 (or other wearable component) and/or neurostimulator module 20 may include one or more additional components. For example, one or more accelerometers or other motion sensors (not shown) may be provided in the housing 22 that are coupled to the controller 24 for detecting motion of the user, e.g., as described elsewhere herein. In addition or alternatively, memory (not shown) may be provided within the housing 22 for storing control parameters and/or data from the controller 24 related to use of the device 10. In another option, an inductive charger (not shown) may be provided in the housing 22 that is coupled to the battery 26 for wirelessly recharging the battery 26, or a connector (also not shown) may be provided on the housing 22 for receiving a cable that may be connected to an external charging device (also not shown).

Optionally, one or more pressure sensors may be provided on the ring 12 that may be coupled to the controller 24 when the module 20 is attached to the ring 12, e.g., to measure dilation forces applied to the ring 12 by the user's penis, measure rigidity of the penis, and/or to measure contact pressure between the user and their partner's body. In another option, a pulse oximetry or other sensor may be provided on the ring 12 (also coupled to the controller 24), e.g., to measure pulse rate, oxygen levels, and/or other parameters of the user. As described elsewhere herein, signals from these sensors may be monitored and/or analyzed by the controller 24 during use, e.g., to monitor the user's response and/or to change parameters of stimulation during use.

In addition, as shown in FIG. 1C, the ring 12 and housing 22 may include one or more cooperating connectors 13, 23 to allow the neurostimulator module 20 to be removably attached to the ring 12, e.g., using one or more detents, snaps, adhesives, hook and eye fasteners, and the like (not shown). Thus, a ring 12 may be used multiple times with the neurostimulator module 20 and, after a predetermined or desired number of uses, the ring 12 may be replaced and the neurostimulator module 20 connected to the new ring for further use. In addition, the connectors 13, 23 may allow the neurostimulator module 20 to be removed for recharging when not in use. Alternatively, the neurostimulator module (or individual components thereof) may be permanently integrated into the ring 12, e.g., by bonding with adhesive, fusing, heat sealing, and the like, and the entire device 10 may be replaced when warranted. In this alternative, the material of the ring 12 (or other wearable component) may be sufficiently durable to last for the life of the device 10, e.g., without substantial wear during use, cleaning, and the like.

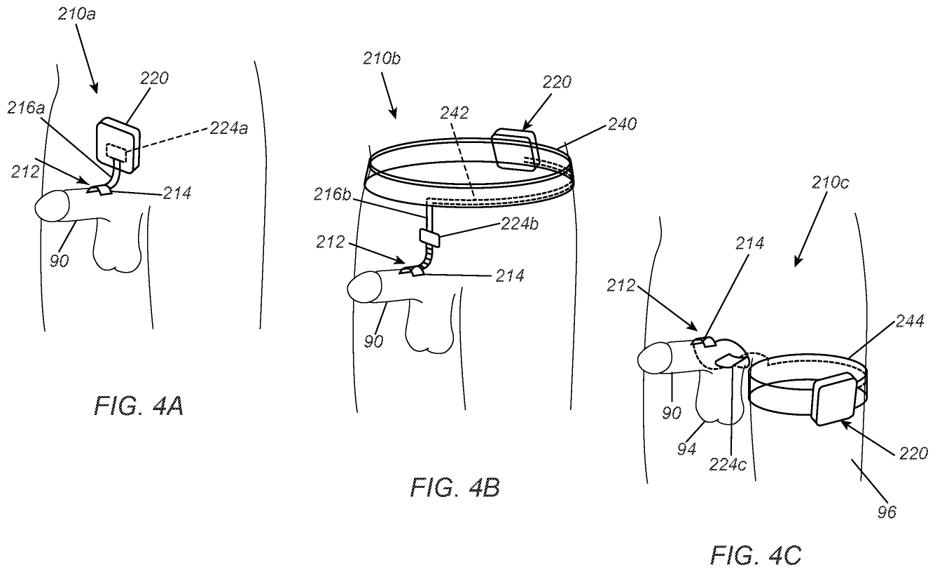

In another alternative, the neurostimulator module may be provided separate from the wearable component and connected to the electrode(s) (and any other optional components provided on the ring) via one or more wires or using remote signaling. For example, as shown in FIGS. 4A-4C, the neurostimulator module may be a separate remote control unit 220, which may be held by the user or worn, e.g., on the user's belt, underwear, and/or other article of clothing, e.g., secured by a clip or other connector (not shown). In this alternative, the control unit 220 may be recharged between uses, e.g., using inductive charging or by connecting a charging cable (not shown) to the control unit 220.

For example, as shown in FIG. 4A, a device 210 is shown that includes a wearable component 212 (which may be any of the embodiments herein) including one or more electrodes 214a contacting the penis 90 when attached to the penis 90. The control unit 210 may be placed against the user's skin, e.g., immediately above or otherwise near the penis 90, and secured, e.g., using low tack adhesive, one or more straps (not shown), and the like. In this alternative, a cable 216a including one or more wires (not shown) may extend from the control unit 210 to the wearable component 212. Ends of the cable 216a may be permanently attached to one or both of the control unit 220 and the wearable unit 212 (and coupled to the electrode(s) 214a by one or more leads on the wearable unit 212) or a connector may be provided on one or both such that the cable 216 may be disconnected when the device 210 is not in use. Optionally, as shown, the device 210 may include a second set of electrodes 224a (including one or more electrodes) on the control unit 220 that contact the user's skin when the control unit 220 is secured. Thus, in this alternative, the neurostimulator module 220 may include an adhesive patch or other feature for removable fixing the neurostimulator module to the user's body, e.g., remote from the user's penis 90, with one or more wires 216a extending to the wearable component to deliver the desired electrical stimulation or via a wireless stimulation.

In a further alternative device 210b, shown in FIG. 4B, instead of an adhesive patch, the neurostimulator module 220 may be carried on a belt 240 that may be secured around the user's torso. In this alternative, one or more wires 242 may be provided in the belt 240 that are coupled to cable 216b that is coupled, in turn, to the electrode(s) 214 on the wearable component 212. FIG. 4C shows another alternative device 210c in which the neurostimulator module 220 may be carried on a garter belt 244 or similar support that may be secured around the user's leg 96. Either belt 240, 244 may include one or more straps and/or connectors for securing ends of the straps together, e.g., using one or more of buckles, hook and eye fasteners, and the like (not shown), to allow the belt 240, 244 to be tightened and/or otherwise secured to the user's body before use and then removed after use. The neurostimulator module 220 may be received in a pocket (not shown) and/or otherwise removably secured to the belt 240, 244 or permanently attached to the belt 240, 244, as desired.

Optionally, the devices 210b, 210c includes a second electrode (or set of electrodes) 224b, 224c that are connected to cable 216b, 216c such that the second electrode(s) 224b, 224c may be placed at a desired location on the user's body, e.g., above the penis 90, as shown in FIG. 4B, or to the perineum behind the scrotum 94, as shown in FIG. 4C. The second electrode(s) 224b, 224c may be attached to the user's skin, e.g., using a low tack adhesive or other feature allowing removal after use.

With reference to the device 10 shown FIGS. 1A-1C (although applicable to all of the embodiments described herein), the controller 24 may be coupled to the one or more electrodes 14, e.g., via one or more leads 16, for controlling delivery of electrical signals to the electrodes 14 to stimulate a desired region of the user's penis. In an exemplary embodiment, the controller 24 may be configured to deliver relatively low frequency electrical pulses to the penis, e.g., at a frequency between about three and twenty Hertz (3-20 Hz). In further exemplary embodiments, the pulses may have pulse widths between about fifty and two hundred microseconds (50-200 μs), amplitudes between about one and fifty milliamps (1-50 mA), and/or voltages between about one and twenty five Volts (1-25 V). The pulses may have substantially uniform parameters, e.g., within these exemplary ranges, or the parameters may be varied, e.g., varying the pulse widths and/or varying the strength (e.g., amplitudes) in a cyclical or other desired pattern to enhance stimulation, as described elsewhere herein. In an exemplary embodiment, the device 10 may deliver a sinusoidal or other crescendo/decrescendo cyclical pattern having desired durations, e.g., between about two and sixty second (2-60 sec) durations. The cyclical patterns may be continuous once the device 10 is activated or the patterns may be separated by gaps having desired durations. In addition or alternatively, the patterns may be modified by the controller 24 based on one or more factors, e.g., feedback from sensors of the device 10, instructions from a remote control unit or electronic device, and the like, as described further elsewhere herein. Optionally, the pulses may be delivered in a bipolar or monopolar configuration between the electrodes.

The communications interface 28 may be coupled to and/or otherwise communicate with the controller 24 for receiving external commands related to delivery of the electrical stimulae. For example, a wireless radiofrequency interface may be provided for receiving wireless signals, e.g., using Bluetooth or other radiofrequency communications protocols, including commands related to delivery of the electrical stimulae. The command may simply include an activation command, whereupon the controller 24 may deliver a preset pattern or sequence of electrical stimulate and then discontinue delivery and wait for further instructions, or the commands may include activation/deactivation instructions. Alternatively, the commands may include instructions regarding the parameters of the pulses and/or repeating pulse delivery one or more times, e.g., parameters selected by the user, modified parameters based on feedback from sensor(s) on the device 10, and the like, as described further elsewhere herein.

In an exemplary embodiment, as shown in FIG. 2A, the interface 28 may allow the device 10 to communicate with a remote electronic device 30, e.g., a dedicated remote control unit, the user's mobile phone, tablet computer, and the like, which may include a user interface 32 (e.g., including one or more of a display, touchscreen, keyboard, and the like) and a communication interface (not shown), e.g., using Bluetooth or other wireless protocols, as represented by signals 34. For example, a software application may be provided that may be loaded into memory of the electronic device 30 to allow the user to deliver commands to the neurostimulator module 20, e.g., to simply activate/deactivate the device 10 and/or to select preset or custom patterns or sequences of electrical stimulae.

During use, as shown in FIG. 1, the ring 12 (or other wearable component described herein) may be placed on the user's penis 90 to position one or more electrodes 14 at a desired region on the penis 90. For example, as shown in FIGS. 2A and 2B the user may position the ring 12 at the base of the penis 90, e.g., to deliver electrical stimulae to the paired dorsal nerves 92, to the dorsal/ventral phallus, and/or to the lateral phallus. Optionally, a conductive gel or other liquid may be applied, e.g., directly to the penis 90 and/or to the inner surface 12a of the ring 12, to enhance electrical coupling between the electrodes 14 and the user's skin.

Optionally, one or more additional electrodes may be provided that are coupled to the device 10 that may be attached to other regions of the user's body. For example, one or more electrodes may be attached to the user's perineum, scrotum, abdomen, pelvis, and/or other locations, as shown and described with reference to FIGS. 4A-4C. Alternatively, a separate device including electrodes and a neurostimulator (not shown) may be provided that may be secured to the perineum or other region to deliver stimulation to the perineum or other region of the user's body.

Once the ring 12 (or other wearable component described herein) is properly positioned, the neurostimulator module 20 may be activated to deliver electrical stimulae to the desired region via the one or more electrodes 14 in a desired manner, e.g., as described elsewhere herein.

For example, it will be appreciated that the device 10 may be used to deliver electrical stimulae to induce and/or expedite orgasm in a variety of ways. For example, the device 10 may be placed on the user's penis 90 and used to deliver electrical stimulae during sexual stimulation, e.g., intercourse or other sexual activity. Alternatively, the device 10 may be used prior to sexual stimulation, e.g., anywhere from five minutes to twenty four hours before sexual activity, e.g., with the device 10 delivering various sequences of electrical stimulae. In another alternative, the device 10 may be used daily and/or at other periodic intervals that are not related to the user's sexual activity. Usage time during any particular session may be the same or variable depending on the user's preferences, e.g., between about three and thirty minutes per session.

With additional reference to FIG. 2A, the wearable device 10 (or any of the devices described herein) may be included in a system 8, e.g., including a dedicated remote control or other electronic device 30, e.g., a mobile phone, tablet, and the like, that may communicate with the device 10 remotely to assist the user in inducing or expediting orgasm. For example, the user may install a software application on their existing electronic device 30 that may allow the user to control the neurostimulator module 20 during use. Optionally, the application may allow the electronic device 30 to receive data from the neurostimulator module 20, e.g., to allow the user or a medical or other professional to monitor response of the user and/or adjust parameters of the neurostimulator module 20.

Alternatively, the device 10 may include a dedicated remote control including a user interface and electronics dedicated to operation of the device 10. The remote control may be a simple device, e.g., including a switch that the user may actuate to turn the device 10 on and off, e.g., with the controller 24 delivering electrical stimulae immediately upon activation of the device 10. Alternatively, the remote control may provide a more sophisticated user interface to allow the user to send instructions to the neurostimulator module 20.

In an exemplary embodiment, the controller 24 may be preprogrammed with one or more stimulae parameters or patterns that may be used initially to deliver electrical stimulae to the penis 90. After using different available patterns, the user may provide feedback, e.g., via the user interface 32 of the electronic device 30 (or remote control). The processor (not shown) of the electronic device 30 (or a server or other system communicating with the electronic device 30) may modify parameters or patterns based on the feedback and send instructions to the controller 24 of the neuro stimulator module 20 to modify parameters for future electrical stimulae accordingly. Optionally, a menu of available parameters may be presented on the user interface 32 of the electronic device 30 and the user may select desired parameters before or during use and corresponding instructions may be communicated to the controller 24.

Optionally, if the device 10 includes one or more sensors, the controller 24 may process signals from the sensors to monitor one or more parameters of the user, and modify patterns of the electrical stimulae based at least in part on the signals. Alternatively, the controller 24 may communicate the signals and/or parameters to the electronic device 30 for processing and/or determining how to modify the patterns used by the controller 24. For example, if a motion sensor is provided on the device 10, the controller 24 (or electronic device 30) may modify the pattern of electrical stimulae when motion is detected, e.g., rapid or cyclical motion, which may indicate sexual activity, whereupon the intensity of the electrical stimulae may be increased, e.g., to induce or expedite orgasm. In addition or alternatively, other parameters of the user may be monitored, e.g., pulse rate, oxygen concentration, pressure, and the like, which may be used in a similar manner to modify patterns and/or intensity of the electrical stimulae. Optionally, the controller 24 (and/or the electronic device 30) may suggest adjustments to the patterns of stimulae and communicate them to the electronic device 30 for presentation and/or selection by the user. Optionally, via communications with a central server, settings and/or feedback with multiple users may be compiled and/or analyzed, e.g., using a machine learning platform that may optimize device settings based on patient preferences/sensory inputs.

In another option, the controller 24 may use the electrodes 14 to detect one or more physical parameters of the user. For example, the controller 24 may receive signals from the electrodes 14 corresponding to muscular contraction, motion, and the like, which may be processed by the controller 24 or communicated to the electronic device 30 for processing, e.g., for diagnostic or other purposes.

Optionally, the application on the electronic device 30 (or dedicated remote control) may provide a training program to identify and/or assist the user in selecting stimulation treatments that may condition the user for quicker orgasm, e.g., for future use with or without the device 10.

While the devices, systems, and methods described herein have been described with reference to expediting or inducing orgasm, e.g., in subjects experiencing delayed orgasm, the devices, systems, and methods herein may be used to treat other conditions, such as premature ejaculation, rehabilitation after pelvic cancer/injury, and the like, or to assist in diagnostic procedures, such as electromyography (“EMG”) and the like, involving the penis. For example, after a patient undergoes pelvic cancer treatment (e.g. surgery or radiation), the devices and systems herein may be used to stimulate and/or rehabilitate penile (and or pelvic) sensory pathways.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A device for inducing or expediting orgasm, comprising:
a wearable component sized for placement around a penis, the wearable component including one or more electrodes for delivering electrical stimulae to the penis; and
a neurostimulator module comprising:
a controller coupled to the one or more electrodes for controlling delivery of the electrical stimulae; and
a communications interface coupled to the controller for receiving external commands related to delivery of the electrical stimulae.

2. The device of claim 1, wherein the neurostimulator module is removably mountable to the wearable component such that, when mounted on the wearable component, the controller is coupled to the one or more electrodes.

3. The device of claim 1, wherein the neurostimulator module is permanently fixed to the wearable component.

4. The device of claim 1, wherein the wearable component comprises a tubular sleeve including open ends or an open end and a closed end and wherein the one or more electrodes are fixed to an inner surface of the tubular sleeve.

5. The device of claim 1, wherein the wearable component comprises a plurality of rings or bands connected together by one or more supports.

6. The device of claim 1, wherein the wearable component comprises a wearable ring, and wherein the one or more electrodes are provided on an inner surface of the ring.

7. A device for inducing or expediting orgasm, comprising:
a wearable ring sized for placement around a penis, the ring including one or more electrodes on an inner surface of the ring for delivering electrical stimulae to the penis; and
a neurostimulator module comprising:
a controller coupled to the one or more electrodes for controlling delivery of the electrical stimulae; and
a communications interface coupled to the controller for receiving external commands related to delivery of the electrical stimulae.

8. The device of claim 7, wherein the neurostimulator module is removably mountable to the ring such that, when mounted on the ring, the controller is coupled to the one or more electrodes.

9. The device of claim 7, wherein the ring comprises an enclosed band formed from elastic material such the band is biased to a relaxed state but is stretchable to accommodate placement and removal or to adjust in size when the penis is stimulated.

10. (canceled)

11. The device of claim 7, wherein the ring has a “C” shaped cross-section including first and second ends.

12. The device of claim 11, wherein the ring is formed from elastic material such the ring is biased to a relaxed state but is stretchable to accommodate placement and removal or to adjust in size when the penis is stimulated.

13. The device of claim 12, wherein the first and second ends are spaced apart from one another in the relaxed state.

14. The device of claim 12, wherein the first and second ends overlap one another in the relaxed state.

15. The device of claim 11, wherein the ring is malleable such that the first and second ends may be moved relative to one another to adjust an inner diameter of the ring.

16. The device of claim 7, wherein the ring comprises a helical ring that spirals one or more times around a central axis between opposite ends of the helical ring.

17-18. (canceled)

19. The device of claim 7, wherein the one or more electrodes comprise a pair of electrodes positioned adjacent one another on the inner surface.

20. The device of claim 7, wherein the one or more electrodes comprise a plurality of electrodes spaced apart from one another on the inner surface around a circumference of the ring.

21. The device of claim 1, wherein the neurostimulator module is provided within a remote control device and wherein a cable including one or more wires electrically couples the controller to the one or more electrodes.

22-40. (canceled)

41. A system for inducing or expediting orgasm, comprising:
a wearable component sized for placement around a penis, the wearable component including one or more electrodes for delivering electrical stimulae to the penis; and
a neurostimulator module comprising:
a controller coupled to the one or more electrodes for controlling delivery of the electrical stimulae; and
a communications interface coupled to the controller for receiving external commands related to delivery of the electrical stimulae; and
an electronic device comprising a user interface and communications interface to allow a user to send commands to the neurostimulator module.

42-50. (canceled)

51. A method for inducing or expediting orgasm, comprising:
placing a wearable device on or adjacent a penis to position one or more electrodes at a desired region relative to the penis; and
activating a neurostimulator on the wearable device to deliver electrical stimulae to the desired region via the one or more electrodes.

52-65. (canceled)
